(19) United States
(12) Patent Application Publication
(10) Pub. No.: US 2026/0297029 A1
(43) Pub. Date: Oct. 1, 2026

(54) ALKOXY AND CARBAMOYL QUATERNARY AMINE SALTS AS PRODRUGS OF TRYPTAMINES

(71) Applicant: Atai Therapeutics, Inc., New York, NY (US)

(72) Inventors: Alan GIBBS, Wyndmoor, PA (US); Tanweer KHAN, Berlin (DE); Robert B PERNI, Marlborough, MA (US); Glenn SHORT, Encinitas, CA (US)

(21) Appl. No.: 19/478,315

(22) PCT Filed: Apr. 29, 2024
(86) PCT No.: PCT/US2024/026797

§ 371 Date: Oct. 24, 2025

Related U.S. Application Data
(60) Provisional application No. 63/576,462, filed on Apr. 27, 2023.

(51) Int. Cl. C07D 209/16; A61K 31/4045; A61K 31/454; A61K 31/5377; C07D 401/12; C07D 403/12

(52) U.S. Cl. CPC C07D 209/16; A61K 31/4045; A61K 31/454; A61K 31/5377; C07D 401/12; C07D 403/12

(57) ABSTRACT

Pharmaceutical compounds with potential for treating mental health disorders which overcome solubility and oral bioavailability issues to act as (partial) agonists of the CNS serotonin receptor, 5-HT2A.

### FIELD OF THE INVENTION

The present disclosure relates to novel compounds with potential for treating mental health disorders which overcome solubility and oral bioavailability issues to act as (partial) agonists of the CNS serotonin receptor, 5-HT2A. Specifically, these compounds persist through first pass metabolism and cross the blood brain barrier, resulting in therapeutically effective concentrations at the site of action.

### BACKGROUND OF THE INVENTION

G protein-coupled receptors, or GPCRs, are a major class of membrane proteins. Approximately 800 different GPCRs are encoded by the human genome and when expressed are located in the plasma membrane to act as the ‘eyes and ears’ of the cell (Gurevich and Gurevich, GPCR Signaling Regulation: the Role of GRKs and Arrestins, Front Pharmacol 10:125 (2019)). Structurally they are composed of seven transmembrane alpha helices connected by intra- and inter-cellular loops of various lengths. These helices and loops play important roles in binding effectors, and/or other proteins, which often results in a signaling or communication event. Signaling of GPCRs produce cellular responses crucial for the health and benefit of the cell and organism.

Several GPCRs are expressed in the central nervous system (CNS), one example is the serotonin family of receptors. The serotonin family is divided into subfamilies, 5-HT1 to 5-HT7 (note: 5-HT3 is a non-GPCR subfamily) and further into subtypes, eg.: the 5-HT2 subfamily is composed of 5-HT2A, 5-HT2B and 5-HT2C (Pandy-Szekeres, G. et. al., The G protein database, GprotieinDb. Nucleic acids research, 50:D518-D525 (2022)). 12 serotonin GPCR subtypes have been identified. Serotonin receptors bind serotonin (or 5-hydroxytryptamine) triggering signal transduction, the downstream effects of which modulate a variety of processes such as: memory, sleep, mood and vision among others (Sizemore, T. R., et. al., Serotonergic modulation across sensory modalities, J Neurophysiol, 123, 2406 (2020)). In addition to serotonin, serotonin receptors are known to bind other endogenous neurotransmitters as well as exogenous small molecules. Indeed, many small molecule drugs have been developed that either activate or deactivate serotonin receptors leading to positive outcomes for a variety of neuropsychiatric disorders (Terry, A. V., Drugs that target serotonergic receptors. In: Buccafusco, J. J. (eds) Cognitive Enhancing Drugs. Milestones in Drug Therapy MDT. Birkhäuser, Basel. https://doi.org/10.1007/978-3-0348-7867-8_6).

Compounds such as the classic psychedelic tryptamines including N, N-dimethyltryptamine (DMT), 5-methoxy-DMT (5-MeO-DMT) and psilocybin (specifically psilocin or 4-hydroxy-DMT) bind select serotonin receptors in the active state and are known to be agonists or partial agonists (McClure-Begley, T. D and Roth, B. L., The promises and perils of psychedelic pharmacology for psychiatry. Nat Rev Drug Discov 21, 463-473 (2022)). These compounds have attracted increasing attention as they are thought to be therapeutically efficacious for various mental health disorders such as MDD, TRD, SUD, as well as compulsive, anxiety, stress and eating disorders (Mertens, L. J. and Preller, K. H., Classical Psychedelics as Therapeutics in Psychiatry-Current Clinical Evidence and Potential Therapeutic Mechanisms in Substance Use and Mood Disorders. Pharmacopsychiatry, 54 (4). 176 (2021)). However, many classic psychedelic tryptamines suffer from poor oral bioavailability due to metabolism by monoamine oxidases (MAOs) and are therefore not suitable as oral therapeutic agents. For example, a strong first pass effect oxidizes 100% of DMT after oral administration and therefore DMT must be administered with a MOA-A inhibitor to be orally active (Riba, J., et. al., Metabolism and urinary disposition of N, N-dimethyltryptamine after oral and smoked administration: a comparative study. Drug Test Anal, 7 (5), 401 (2015)). Furthermore, classic psychedelic tryptamine solubility and duration of target engagement may not be ideal for many therapeutic purposes.

Therefore, a significant need exists for readily administrable medications which overcome solubility and oral bioavailability issues to act as (partial) agonists of the CNS serotonin receptor, 5-HT2A, to treat neurological diseases and conditions.

### SUMMARY OF THE INVENTION

The present disclosure provides, in one respect, compounds which act as (partial) agonists of the central nervous system (CNS) serotine receptor, 5-HT2A. These compounds overcome solubility and oral bioavailability issues and persist through first pass metabolism and cross the blood brain barrier. The compounds identified herein maintain overall selectivity profiles similar to that of DMT, 5-MeO-DMT and 4-hydroxy-DMT but with potentially improved PK properties and half-life.

In some embodiments, the compounds are a compound of Formula I:

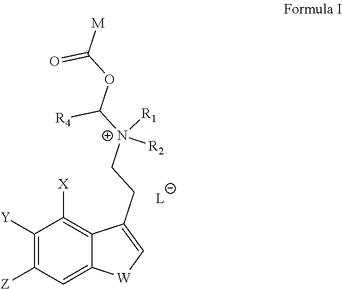

wherein L is a counter ion;
X, Y, and Z are independently hydrogen, deuterium, OR₁, CO₂R₁, or NR₁R₂;
W is NR₁, OR₁, or CR₁R₂;
M is a CR₅R₆R₇, OR₅, or NR₅R₆; and
R₁, R₂, R₄, R₅, R₆, and R₇, are independently hydrogen, deuterium, alkyl, cycloalkyl, heteroatom substituted and halogen substituted alkyl or cycloalkyl, aromatic, or heteroaromatic.

In some embodiments, L is a Cl, Br, I, CH₂FCOO, CF₃COO, C₁-C₆ aliphatic carboxylic acid anion, or C₁-C₆ aliphatic sulfonic acid anion.

In some embodiments, R₁ and R₂ and/or R₅ and R₆ are combined in a 3-7 membered saturated ring.

In some embodiments, the compound has a solubility greater than 200 μM.

In some embodiments, the compound has a half-life greater than 1,500 minutes in simulated gastric fluid.

In some embodiments, the compound has a half-life greater than 1,500 minutes in simulated gastric fluid with pepsin.

In some embodiments, the compound has a half-life greater than 1,500 minutes in PBS (pH 6.5).

In some embodiments, the compound further comprises a pharmaceutical carrier.

In some embodiments, the compound is administered to a patient orally.

In some embodiments, the compound is used to treat a neurological disease or condition.

In some embodiments, the compound is synthesized by:
reacting the tryptamine solution with Formula II:

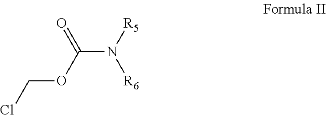

wherein R₅ and R₆ is independently alkyl, cycloalkyl, heteroatom substituted or halogen substituted alkyl or cycloalkyl, aromatic or heteroaromatic.

In some embodiments, the compound is synthesized by:
reacting a tryptamine solution with Formula III:

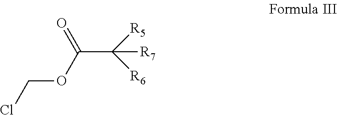

wherein R₅, R₆, and R₇ is independently hydrogen, deuterium, alkyl, cycloalkyl, heteroatom substituted or halogen substituted alkyl or cycloalkyl, aromatic or heteroaromatic.

### DETAILED DESCRIPTION

Compounds that overcome solubility and oral bioavailability issues with potential for treating mental health disorders are identified herein. These compounds act as (partial) agonists of the CNS serotonin receptor, 5-HT2A, to treat neurological diseases and conditions. These compounds persist through first pass metabolism and cross the blood brain barrier, resulting in therapeutically effective concentrations at the site of action. Moreover, compounds identified herein maintain overall selectivity profiles similar to that of DMT, 5-MeO-DMT, and 4-hydroxy-DMT but with potentially improved PK properties and half-life. The present invention contains, but is not limited to, so-called anti- or soft-drugs. The soft-drugs described herein are (partial) agonists in present form but are subsequently metabolized in vivo to known inactive metabolites, in an appropriate time interval. Also, these prodrugs hydrolyzed hydroxy methyl may act (partial) agonists for the serotonin receptors.

The present invention contains, but is not limited to, the compounds of Formula I:

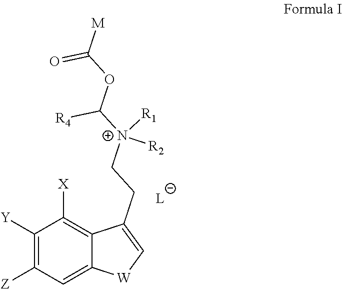

wherein L is a counter ion;
X, Y, and Z are independently hydrogen, deuterium, OR₁, CO₂R₁, or NR₁R₂;
W is NR₁, OR₁, or CR₁R₂;
M is a CR₅R₆R₇, OR₅, or NR₅R₆; and
   R₁, R₂, R₄, R₅, R₆, and R₇ are independently hydrogen, deuterium, alkyl, cycloalkyl, heteroatom substituted and halogen substituted alkyl or cycloalkyl, aromatic, or heteroaromatic.

In one embodiment, L is a Cl⁻, Br⁻, I⁻, CH₂FCOO⁻, CF₃COO⁻, C₁-C₆ aliphatic carboxylic acid anion, or C₁-C₆ aliphatic sulfonic acid anion.

In one embodiment, R₁ and R₂ and/or R₅ and R₆ are combined in a 3-7 membered saturated ring.

Table 1 contains specific compounds of Formula I, Compounds 1-12, that overcome solubility and oral bioavailability issues with potential for treating mental health disorders

TABLE 1
Identified Compounds

| ID | Structure | MW | Name | Smiles |
|---|---|---|---|---|
| 1 | 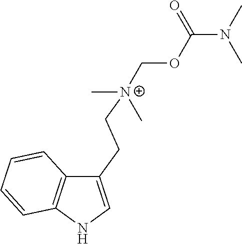 [SMILES: CN(C)C(=O)OC[N+](C)(C)CCc1c[nH]c2ccccc12] | 290.3 | N- (((dimethylcarbamoyl)oxy) methyl)-2-(1H-indol-3-yl)- N,N-dimethylethan-1- aminium | C[N+](C)(CCC1═CNC2═C1 C═CC═C2)COC(N(C)C)═O |
| 2 | 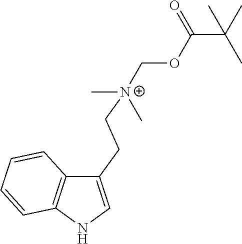 [SMILES: CC(C)(C)C(=O)OC[N+](C)(C)CCc1c[nH]c2ccccc12] | 303.4 | 2-(1H-indol-3-y1)-N,N- dimethy1-N- ((pivaloy loxy)methyl)ethan- 1-aminium | C[N+](C)(CCC1═CNC2═C1 C═CC═C2)COC(C(C)(C)C)═O |
| 3 | 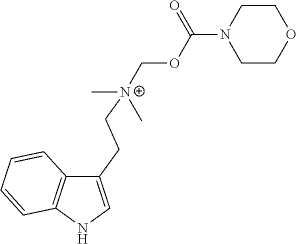 [SMILES: C[N+](C)(CCc1c[nH]c2ccccc12)COC(=O)N1CCOCC1] | 332.4 | 2-(1H-indol-3-y1)-N,N- dimethyl-N-(((morpholine- 4- carbonyl)oxy)methyl)ethan- 1-aminium | C[N+](C)(CCC1═CNC2═C1 C═CC═C2)COC(N3CCOCC3)═ O |
| 4 | 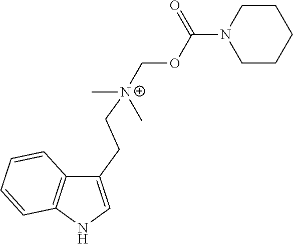 [SMILES: C[N+](C)(CCc1c[nH]c2ccccc12)COC(=O)N1CCCCC1] | 330.4 | 2-(1H-indol-3-y1)-N,N- dimethyl-N-(((piperidine-1- carbonyl)oxy)methyl)ethan- 1 -aminium | C[N+](C)(CCC1═CNC2═C1 C═CC═C2)COC(N3CCCCC3)═ O |
| 5 | 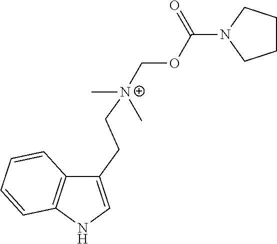 [SMILES: C[N+](C)(CCc1c[nH]c2ccccc12)COC(=O)N1CCCC1] | 316.4 | N-(((cyclopentanecarbonyl) oxy)methyl)-2-(1H-indol-3- yl)-N.N-dimethylethan-1- aminium | C[N+](C)(CCC1═CNC2═C1 C═CC═C2)COC(N3CCCC3)═ O |
| 6 | 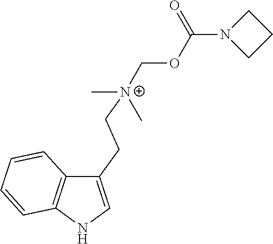 [SMILES: C[N+](C)(CCc1c[nH]c2ccccc12)COC(=O)N1CCC1] | 302.4 | N-(((azetidine-1- carbonyl)oxy)methyl)-2- (1H-indol-3-y1)-N,N- dimethylethan-1-aminium | C[N+](C)(CCC1═CNC2═C1 C═CC═C2)COC(N3CCC3)═O |
| 7 | 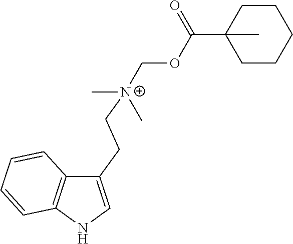 | 343.4 | 2-(1H-indol-3-yl)-N,N- dimethyl-N-(((1- methylcyclohexane-1- carbonyl)oxy)methyl)ethan- 1-aminium | C[N+](C)(CCC1═CNC2═C1 C═CC═C2)COC(C3(C) CCCCC3)═O |
| 8 | 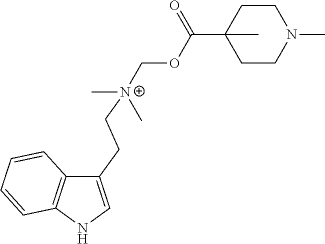 | 358.5 | N-(((1,4- dimethylpiperidine-4- carbonyl)oxy)methyl)-2- (1H-indol-3-y1)-N,N- dimethylethan-1-aminium | C[N+](C)(CCC1═CNC2═C1 C═CC═C2)COC(C3(C)CCN (C)CC3)═O |
| 9 | 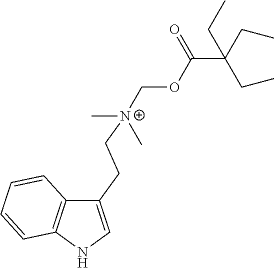 [SMILES: CCC(CC)(CC)C(=O)OC[N+](C)(C)CCc1c[nH]c2ccccc12] | 345.5 | N-(((2,2-diethylbutanoyl) oxy)methyl)-2-(1H-indol- 3-y1)-N,N-dimethylethan- 1-aminium | C[N+](C)(CCC1═CNC2═C1 C═CC═C2)COC(C(CC)(CC) CC)═O |
| 10 | 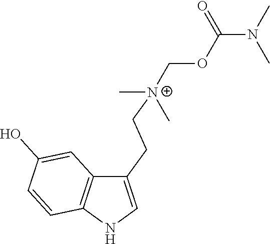 [SMILES: CN(C)C(=O)OC[N+](C)(C)CCc1c[nH]c2ccc(O)cc12] | 306.3 | N- (((dimethylcarbamoyl)oxy) methyl)-2-(5-hydroxy-1H- indol-3-y1)-N,N- dimethylethan-1-aminium | C[N+](C)(CCC1═CNC2═C1 C═C(O)C═C2)COC(N(C)C)═O |
| 11 | 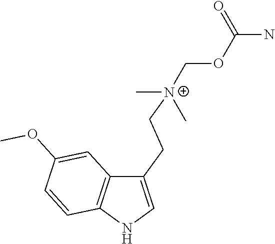 [SMILES: COc1ccc2[nH]cc(CC[N+](C)(C)COC(N)=O)c2c1] | 320.4 | N- (((dimethylcarbamoyl)oxy) methy1)-2-(5-methoxy-1H- indol-3-y1)-N,N- dimethylethan-1-aminium | C[N+](C)(CCC1═CNC2═C1 C═C(OC)C═C2)COC(N(C)C)═O |
| 12 | 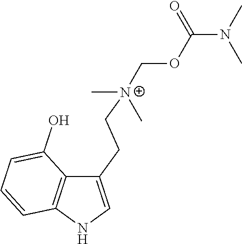 [SMILES: CN(C)C(=O)OC[N+](C)(C)CCc1c[nH]c2cccc(O)c12] | 306.3 | N- (((dimethylcarbamoyl)oxy) methyl)-2-(4-hydroxy-1H- indol-3-yl)-N,N- dimethylethan-1-aminium | C[N+](C)(CCC1═CNC2═C1 C(O)═CC═C2)COC(N(C)C)═O) (CC)CC)═O |

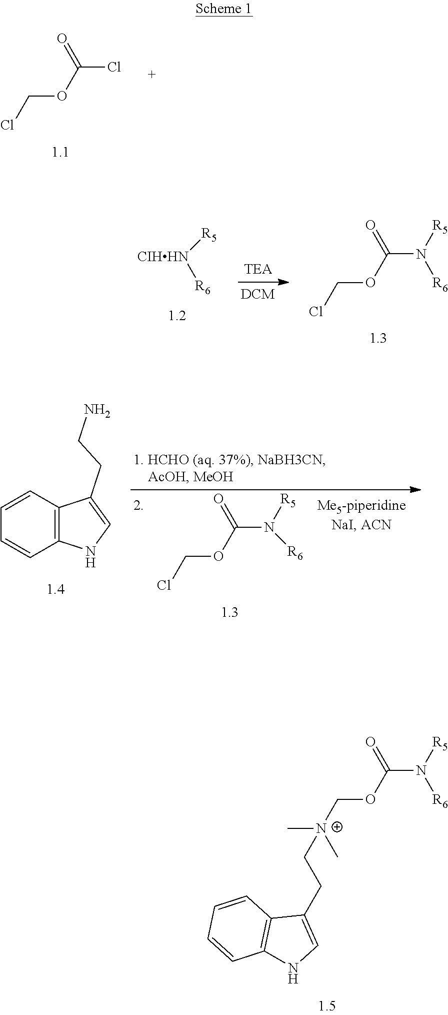

Scheme 1 illustrates the general method of which all carbamoyl amin salts in Table 1 can be synthesized by (Compounds 1, 3-6, and 10-12).

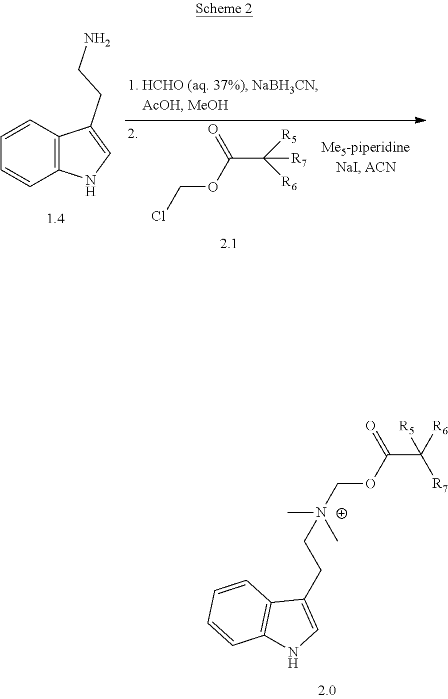

Scheme 2 illustrates the general method of which all alkoxy quaternary amin salts in Table 1 can be synthesized by (Compounds 2 and 7-9).

### Example 1

Compound 1 was synthesized by following the method shown in Scheme 3:

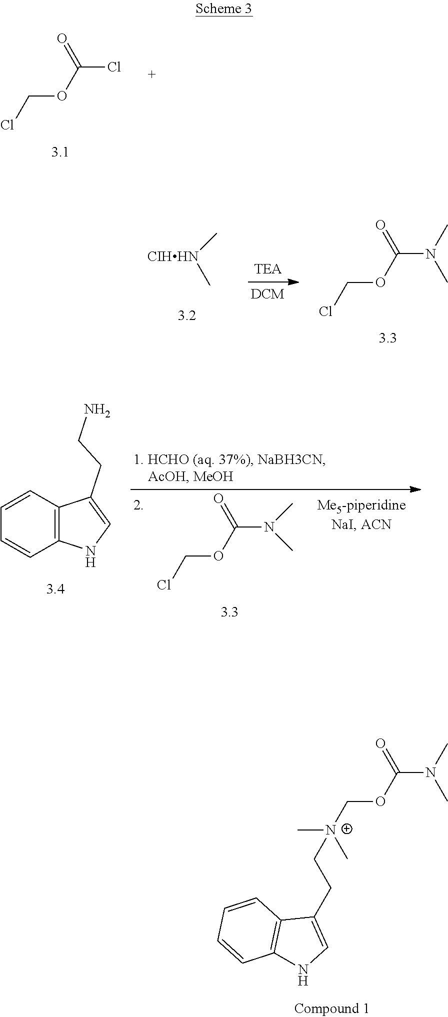

To a stirred mixture of chloromethyl chloroformate 3.1 (1.0 g, 7.7 mmol, 1.0 equiv.) in THF was added TEA (3.9 g, 38.7 mmol, 5.0 equiv.) and dimethylamine 3.2 (0.70 g, 15.5 mmol, 2.0 equiv.) dropwise at −20° C. under an argon atmosphere. The resulting mixture was stirred for an additional 2 h at room temperature. The reaction was quenched by the addition of water (100 mL) and the resulting mixture was extracted with DCM (3×100 mL). The combined organic extracts were washed with saturated sodium bicarbonate aqueous solution (1×300 mL), dried over anhydrous Na₂SO₄, and concentrated under reduced pressure. The crude product. 3.3, chloromethyl dimethylcarbamate was used in the next step directly without further purification.

To a solution of tryptamine (200 mg, 1.25 mmol, 1.0 equiv.) in 3 mL of methanol was added sodium cyanoborohydride (197 mg, 3.13 mmol, 2.5 equiv.), acetic acid (0.15 mL) and formaldehyde (254 mg, 3.13 mol, 2.5 equiv., 37% in aq. solution) in methanol (1.5 mL) at 0° C. The mixture solution was stirred overnight at 25° C. and then concentrated under vacuum. The residue was diluted with 20 mL of saturated sodium bicarbonate and extracted with ethyl acetate (3×20 mL). The combined organic extracts were washed with brine (2×20 mL), dried over anhydrous sodium sulfate, and concentrated under vacuum. The resulting residue was dissolved in 5 mL of acetonitrile and 1,2,2,6,6-pentamethylpiperidine (291 mg, 1.8 mmol, 2.0 equiv.) and NaI (5 mg, 0.03 mmol, 0.1 equiv.) were added followed by 3.3 (193 mg, 1.4 mmol, 1.5 equiv.) portion-wise at room temperature under an argon atmosphere. The resulting mixture was stirred overnight at 40° C. and concentrated under vacuum. The residue was purified by reversed-phase flash chromatography; mobile phase, MeCN in water, 10% to 50% gradient over 10 min. The product (53 mg) was further purified by reverse phase Preparative-HPLC; Mobile Phase A: Water (0.1% FA), Mobile Phase B: ACN; Flow rate: 60 mL/min; Gradient: 25% B to 55% B in 8 min, to afford 12 mg of Compound 1, N-(((dimethylcarbamoyl)oxy)methyl)-2-(1H-indol-3-yl)-N,N-dimethylethan-1-aminium. 1H NMR: (400 MHZ, DMSO-d6) δ 11.07 (s, 1H), 8.50 (s, 1H), 7.61 (d, J=8.0 Hz, 1H), 7.38 (d, J=8.0 Hz, 1H), 7.25 (d, J=2.0 Hz, 1H), 7.11-7.03 (m, 2H), 5.35 (s, 2H), 3.60-3.51 (m, 2H), 3.21-3.16 (m, 8H), 2.96 (s, 3H), 2.91 (s, 3H). MS m/z [M]+ (ESI): 290.15.

### Example 2

Compound 2 was synthesized by following the method shown in Scheme 4:

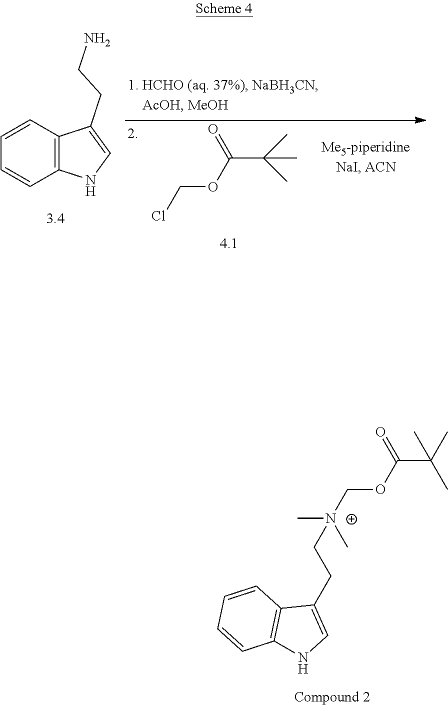

To a solution of tryptamine (200 mg, 1.25 mmol, 1.0 equiv.) in 3 mL of methanol was added sodium cyanoborohydride (197 mg, 3.13 mmol, 2.50 equiv.), acetic acid (0.15 mL) and formaldehyde (254 mg, 3.13 mol, 2.5 equiv., 37% in aq. solution) in methanol (1.5 mL) at 0° C. The mixture solution was stirred overnight at 25° C. and then concentrated under vacuum. The resulting solution was diluted with 20 mL of saturated sodium bicarbonate and extracted with ethyl acetate (3×20 mL). The combined organic layers were washed with brine (2×20 mL), dried over anhydrous sodium sulfate, and concentrated under vacuum. The resulting mixture was dissolved in 5 mL of acetonitrile, 1,2,2,6,6-pentamethylpiperidine (388 mg, 2.5 mmol, 2.0 equiv.) and NaI (14 mg, 0.1 mmol, 0.1 equiv.) were added, followed by chloromethyl 2,2-dimethylpropanoate. 4.1 (282 mg, 1.9 mmol, 1.5 equiv.) portion-wise at room temperature under an argon atmosphere. The resulting mixture was stirred overnight at 40° C. The residue was purified by reversed-phase flash chromatography: mobile phase, MeCN in water (0.1% FA), 5% to 100% gradient over 30 min; 30 mg of Compound 2, 2-(1H-indol-3-yl)-N,N-dimethyl-N-((pivaloyloxy)methyl) ethan-1-aminium was obtained. 1H NMR: (400 MHZ, DMSO-d6) § 11.03 (s, 1H), 7.61 (d, J=7.6 Hz, 1H), 7.38 (d, J=8.0 Hz, 1H), 7.26 (d, J=2.0 Hz, 1H), 7.14-7.10 (m, 1H), 7.05-7.01 (m, 1H), 5.40 (s, 2H), 3.61-3.57 (m, 2H), 3.21-3.17 (m, 8H), 1.25 (s, 9H). MS m/z [M]+ (ESI): 303.15.

### Example 3

Stability testing of Compound 1 and Compound 2 along with control compounds chlorambucil and erythromycin is shown in Table 2. The half-life of the compounds was measured in PBS (pH 6.5), simulated gastric fluid (SGF) with pepsin, and SGF without pepsin.

TABLE 2
Compound Stability Testing

| | | T_{1/2} | Remaining Percentage (%) | | | | |
|---|---|---|---|---|---|---|---|
| Compound | Incubation | (min) | 0 min | 30 min | 60 min | 120 min | 240 min |
| Chlorambucil | PBS (pH 6.5) | 41.63 | 100.00 | 59.41 | 37.13 | 13.58 | 1.83 |
| Erythromycin | SGF with pepsin | 35.98 | 100.00 | 52.60 | 30.48 | 9.74 | 1.00 |
| Erythromycin | SGF without pepsin | 36.62 | 100.00 | 54.48 | 29.11 | 9.44 | 1.05 |
| Compound 2 | PBS (pH 6.5) | 3794.64 | 100.00 | 97.37 | 101.49 | 98.29 | 95.31 |
| Compound 2 | SGF with pepsin | 5325.39 | 100.00 | 94.34 | 104.10 | 96.57 | 96.12 |
| Compound 2 | SGF without pepsin | 9999.00 | 100.00 | 90.71 | 97.28 | 104.20 | 104.48 |
| Compound 1 | PBS (pH 6.5) | 9999.00 | 100.00 | 97.65 | 107.92 | 106.62 | 108.42 |
| Compound 1 | SGF with pepsin | 9999.00 | 100.00 | 100.98 | 100.14 | 97.19 | 108.34 |
| Compound 1 | SGF without pepsin | 15659.89 | 100.00 | 105.39 | 100.25 | 104.17 | 100.27 |

If calculated T_{1/2} < 0, then T_{1/2} was reported as 9999 min.

The large half-life of Compound 1 and Compound 2 in PBS (pH 6.5), SGF with pepsin, and SGF without pepsin indicate that the compound does not degrade in the environment of a stomach nor in the presence of pepsin, therefore persisting through first pass metabolism when orally ingested. This allows for the compounds to enter the bloodstream with little to no loss as an oral dose.

### Example 4

Solubility for Compounds 1 and 2 were measured and is shown in Table 3.

TABLE 3
Compound Solubility

| Compound | SOL (μM) |
|---|---|
| Diclofenac | 275.79 |
| Compound 2 | 313.56 |
| Compound 1 | 289.77 |

Any value close to or above 300 μM indicates that the compound may have a solubility at or above 300 μM.

Solubility measurements were taken according to the steps as followed:

1. Preparation of FaSSIF: Prepare Buffer A by dissolving 1.392 g of NaOH, 2.220 g of Maleic acid and 4.010 g of NaCl into about 900 mL ultrapure water and adjust the pH of the solution to 6.5 with 1 M NaOH. Then dilute the solution with ultrapure water to 1000 mL at room temperature. Add 1.790 g of FaSSIF-V2 Powder to about 500 mL of Buffer A. Stir until powder is completely dissolved. Then dilute the solution with Buffer A to 1000 mL at room temperature. Use within 48 hours at room temperature and within 24 hours at 37° C.

2. Preparation of Stock Solution: The stock solution of test compounds was prepared in dimethyl sulfoxide (DMSO) at the concentration of 30 mM. The stock solution of control compound was prepared in DMSO at the concentration of 30 mM. Diclofenac sodium was used as positive control in the assay.

3. Procedures for Solubility Determination: 10 μL stock solution of each compound was placed in order into their proper 96-well rack, followed by adding 990 μL of FaSSIF into each vial of the cap-less Solubility Sample plate. This study was performed in duplicate. One stir stick was added to each vial and then vials were sealed using a molded PTDE/SIL 96-Well Plate Cover. The Solubility Sample plate was transferred to the Thermomixer Comfort plate shaker and incubated at RT for 2 hours with shaking at 1100 rpm. After 2 hours incubation, stir sticks were removed using a big magnet and all samples from the Solubility Sample plate were transferred into the filter plate. All the samples were filtered by using the Vacuum Manifold. An aliquot of 10 μL of the filtered samples were diluted with 980 μL of methanol and 10 μL of DMSO. The dilution factor might be changed according to the solubility value and the LC/MS signal response.

4. Preparation of 3 μM Standards (STD): DMSO stock solution at 30 mM was diluted with DMSO to 300 μM. An aliquot of 10 μL was transferred to a new 96-well plate followed by adding of 980 μL of methanol and 10 μL of FaSSIF to obtain 3 μM STDs. The dilution factor might be changed according to the LC/MS signal response.

5. Sample Analysis: Samples were analyzed by LC-MS/MS.

6. Data Analysis: All calculations were carried out using Microsoft Excel. The solution filtered was analyzed and quantified against a standard of known concentration in DMSO using LC coupled with Mass spectral peak identification and quantitation. The solubility values of the test compounds were calculated as follows:

$$[ Sample ] = \frac{{AREA}_{Sample} \times INJ {VOL}_{Std} \times {DF}_{Sample} \times [ STD ]}{{AREA}_{Std} \times INJ {VOL}_{Sample}}$$

with DF as the dilution factor.

### Example 5

Stability of Compound 1 and Compound 2 in mouse plasma was measured by calculating the half-life of each compound as seen in Table 4.

TABLE 4
Compound Stability in Plasma

| | | T_{1/2} | Remaining Percentage (%) | | | | |
|---|---|---|---|---|---|---|---|
| Compound | Species | (min) | 0 min | 15 min | 30 min | 60 min | 120 min |
| Propantheline | Mouse | 32.76 | 100.00 | 86.05 | 67.21 | 35.00 | 8.48 |
| Compound 2 | | 9.41 | 100.00 | 24.37 | 7.66 | 1.13 | 0.00 |
| Compound 1 | | 155.64 | 100.00 | 97.48 | 92.59 | 90.25 | 58.31 |

Note:
For the compounds that showed an initial fast disappearance followed by a slow disappearance, only the time points that were within the initial rate were included in the calculation.

Compound 1 was determined to have a half-life of 155 minutes in mouse plasma and Compound 2 was determined to have a half-life of 9.4 minutes in mouse plasma.

### Definitions

As used herein, the following terms are defined with the following meanings, unless explicitly stated otherwise:

The singular form “a”, “an” and “the” include plural references unless the context clearly dictates otherwise. For example, the term “a pharmaceutically acceptable carrier” may include a plurality of pharmaceutically acceptable carriers, including mixtures thereof.

The term “and/or” is intended to mean either or both of two components of the invention.

The term “subject,” “individual” and “patient” are used interchangeably herein, and refers to a human.

The term “device,” as used herein, refers to an apparatus or system capable of delivering a drug to a patient in need thereof.

The term “in need of treatment” and the term “in need thereof” when referring to treatment are used interchangeably and refer to a judgment made by a caregiver (e.g. physician, nurse, nurse practitioner) that a patient will benefit from treatment.

The terms “treat” and “treatment” refer herein to therapeutic treatment, including prophylactic or preventative measures, wherein the object is to prevent or slow down (lessen) an undesired physiological change associated with a disease or condition. Beneficial or desired clinical results include, but are not limited to, alleviation of symptoms, diminishment of the extent of a disease or condition, stabilization of a disease or condition (i.e., where the disease or condition does not worsen), delay or slowing of the progression of a disease or condition, amelioration or palliation of the disease or condition, and remission (whether partial or total) of the disease or condition. “Treatment” can also mean prolonging survival as compared to expected survival if not receiving treatment. Those in need of treatment include those already with the disease or condition as well as those prone to having the disease or condition or those in which the disease or condition is to be prevented. “Treatment” can, when concerning depression, also include reducing at least one sign or symptom of depression. Examples of a sign or symptom of depression include depressed mood, diminished interest in activities, weight loss or gain, decrease or increase in appetite, insomnia or hypersomnia, psychomotor agitation or retardation, fatigue or loss of energy, feelings of worthlessness or excessive or inappropriate guilt, diminished ability to concentrate or indecisiveness, or suicidal ideation or behavior.

The term “pharmaceutically acceptable” as used herein, refers to a component of a pharmaceutical composition that is compatible with the other ingredients of the formulation and not overly deleterious to the recipient thereof.

The term “carrier” refers to a diluent, adjuvant, excipient, or vehicle with which the therapeutic is administered and includes, but is not limited to, such liquids and powders that are hydrophilic substances, hydrophobic substances and substances that possess both hydrophilic and hydrophobic properties such as emulsifiers.

The term “effective amount” or “therapeutically effective amount” as used herein, refers to the amount of active agent that elicits the biological or medicinal response in a tissue, system, or individual that is being sought by a researcher, healthcare provider or individual.

The term “neurological disease or condition” as used herein, means a disease or condition selected from: a neuropsychiatric disorder, such as depression (including severe depression such as treatment-resistant depression, major depressive disorder and persistent depressive disorder), catatonic depression, a depressive disorder due to a medical condition, postpartum depression, premenstrual dysphoric disorder, or seasonal affective disorder, anxiety, anxiety disorder, social anxiety disorder, general anxiety disorder (GAD), avolition disorder, bipolar disorder (including bipolar I disorder and bipolar II disorder), post-traumatic stress disorder, body dysmorphic disorder, abnormalities of mood or emotion, including the above conditions, dysthymia, schizoaffective disorder, schizophrenia and other psychotic disorders, panic disorder, traumatic stress disorders, phobic disorders, and personality disorders with abnormal mood, such as borderline personality disorder, schizoid and schizotypal disorders and suicide ideation, or rumination/unproductive repetitive thoughts negatively impacting one's behavior/mood/ability to focus, obsessive compulsive disorder. addiction (including substance use disorder such as addiction to nicotine, alcohol, cocaine, opioids, amphetamine, methamphetamine, heroin, morphine, phencyclidine, 3,4-methylenedioxy-methamphetamine, as well as other addictive substances), addictive behavior (including eating, gambling, sex, pornography. videogames, work, exercise, spiritual obsession, self-harm, travel and shopping addiction), eating disorder (including anorexia nervosa, bulimia nervosa and binge eating disorder), and pain (including pain associated with migraine or headache or chronic pain).

As used herein, the term “treatment-resistant depression” or “TRD” means a depressive disorder which does not respond satisfactorily to adequate treatment. TRD is a complex phenomenon influenced by variety in depressive subtypes, psychiatric comorbidity, and coexisting medical illnesses. Although TRD episodes are most commonly associated with major depressive disorder (MOD), they are also seen in the depressed phase of bipolar disorder.

All references, articles, publications, patents, patent publications, and patent applications cited herein are incorporated by reference in their entireties for all purposes. However, mention of any reference, article, publication, patent, patent publication, and patent application herein is not, and should not, be taken as acknowledgment or any form of suggestion that they constitute valid prior art or form part of the common general knowledge in any country in the world.

Other embodiments and uses of the invention will be apparent to those skilled in the art from consideration of the specification and practice of the invention disclosed herein. All references cited herein, including all U.S. and foreign patents and patent applications, are specifically and entirely hereby incorporated herein by reference. It is intended that the specification and examples be considered exemplary only, with the true scope and spirit of the invention indicated by the following claims.

What is claimed is:

1. A compound having the structure of Formula (I): 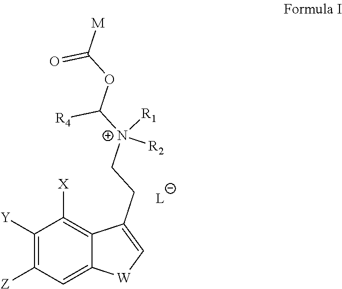
wherein:
L is a counter ion;
X, Y, and Z are independently hydrogen, deuterium, OR₁, CO₂R₁, or NR₁R₂;
W is NR₁, OR₁, or CR₁R₂;
M is a CR₅R₆R₇, OR₅, or NR₅R₆; and
R₁, R₂, R₄, R₅, R₆ and R₇ are independently hydrogen, deuterium, alkyl, cycloalkyl, heteroatom substituted and halogen substituted alkyl or cycloalkyl, aromatic, or heteroaromatic.

2. The compound of claim 1, wherein L is a Cl, Br, I, CH₂FCOO, CF₃COO, C₁-C₆ aliphatic carboxylic acid anion, or C₁-C₆ aliphatic sulfonic acid anion.

3. The compound of claim 1, wherein R₅ and R₆ are combined in a 3-7 membered saturated ring.

4. The compound of claim 1, wherein R₁ and R₂ are combined in a 3-7 membered saturated ring.

5. The compound of claim 1, wherein the compound persists through first pass metabolism of a mammal.

6. The compound of claim 5, wherein the compound acts as a partial agonist of a CNS serotonin receptor.

7. The compound of claim 1, wherein:
X, Y, and Z are hydrogen;
R₁ and R₂ are CH₃;
R₄ is hydrogen;
W is NH; and
M is N(CH₃)₂.

8. The compound of claim 7, wherein the compound has a solubility greater than 200 μM in dimethyl sulfoxide (DMSO).

9. The compound of claim 8, wherein the compound has a solubility between 275 μM and 300 μM in dimethyl sulfoxide (DMSO).

10. The compound of claim 7, wherein the compound has a half-life greater than 100 minutes in plasma.

11. The compound of claim 10, wherein the compound has a half-life between 125 minutes and 175 minutes in plasma.

12. The compound of claim 7, wherein the compound has a half-life greater than 1,500 minutes in simulated gastric fluid.

13. The compound of claim 7, wherein the compound has a half-life greater than 1,500 minutes in simulated gastric fluid with pepsin.

14. The compound of claim 7, wherein the compound has a half-life greater than 1,500 minutes in phosphate-buffered saline (PBS) with a pH of 6.5.

15. The compound of claim 1, wherein:
X, Y, and Z are hydrogen;
R₁ and R₂ are CH₃;
R₄ is hydrogen;
W is NH; and
M is C(CH₃)₃.

16. The compound of claim 15, wherein the compound has a solubility greater than 275 μM in dimethyl sulfoxide (DMSO).

17. The compound of claim 16, wherein the compound has a solubility between 280 μM and 340 μM in dimethyl sulfoxide (DMSO).

18. The compound of claim 15, wherein the compound has a half-life greater than 7 minutes in plasma.

19. The compound of claim 18, wherein the compound has a half-life between 8 minutes and 11 minutes in plasma.

20. The compound of claim 15, wherein the compound has a half-life greater than 1.500 minutes in simulated gastric fluid.

21. The compound of claim 15, wherein the compound has a half-life greater than 1,500 minutes in simulated gastric fluid with pepsin.

22. The compound of claim 15, wherein the compound has a half-life greater than 1,500 minutes in phosphate-buffered saline (PBS) with a pH of 6.5.

23. The compound of claim 1, wherein:
R₁ and R₂ are CH₃;
R₄ is hydrogen; and
W is NH.

24. The compound of claim 23, wherein X, Y, and Z are hydrogens.

25. The compound of claim 24, wherein M is morpholine.

26. The compound of claim 24, wherein M is piperidine.

27. The compound of claim 24, wherein M is pyrrolidine.

28. The compound of claim 24, wherein M is azetidine.

29. The compound of claim 24, wherein M is 1-methylcyclohexane.

30. The compound of claim 24, wherein M is 1,4-dimethylpiperidine.

31. The compound of claim 24, wherein M is 3-ethylpentane.

32. The compound of claim 23, wherein;
M is N(CH₃)₂;
X is H;
Y is OH; and
Z is H.

33. The compound of claim 23, wherein;
M is N(CH₃)₂;
X is H;
Y is OCH₃; and
Z is H.

34. The compound of claim 23, wherein;
M is N(CH₃)₂;
X is OH;
Y is H; and
Z is H.

35. The compound of claim 1, further comprising a pharmaceutical carrier.

36. A method of treating a neurological disease or condition comprising administering to a subject in need thereof the composition of claim 1.

37. The method of claim 36, wherein the compound is administered orally.

38. The method of claim 36, wherein the neurological disease or condition is a neuropsychiatric disorder, premenstrual dysphoric disorder, or seasonal affective disorder, anxiety, anxiety disorder, social anxiety disorder, general anxiety disorder (GAD), avolition disorder, bipolar disorder, post-traumatic stress disorder, body dysmorphic disorder, abnormalities of mood or emotion, dysthymia, schizoaffective disorder, schizophrenia a, panic disorder, traumatic stress disorders, phobic disorders, and personality disorders with abnormal mood, addiction, addictive behavior, eating disorder, or pain.

39. A method of preparing the compound of claim 1, the method comprising:
reacting a tryptamine solution with Formula II: 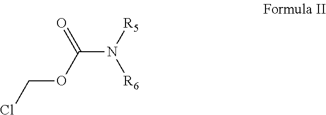
wherein R₅ and R₆ is independently alkyl, cycloalkyl, heteroatom substituted or halogen substituted alkyl or cycloalkyl, aromatic, or heteroaromatic.

40. The method of claim 39, wherein R₅ and R₆ are methyl.

41. The method of claim 39, wherein R₅ and R₆ are combined in a 3-7 membered saturated ring.

42. A method of preparing the compound of claim 1, the method comprising:
reacting a tryptamine solution with Formula III: 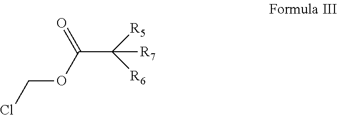
wherein R₅, R₆, and R₇ is independently hydrogen, deuterium, alkyl, cycloalkyl, heteroatom substituted or halogen substituted alkyl or cycloalkyl, aromatic, or heteroaromatic.

43. The method of claim 42, wherein R₅ and R₆ are combined in a 3-7 membered saturated ring.

44. The method of claim 42, wherein R₅, R₆, and R₇ are methyl.
